(19) United States
(12) Patent Application Publication
(10) Pub. No.: US 2009/0183391 A1
(43) Pub. Date: Jul. 23, 2009

(54) RIGID ORTHOTIC WITH RESILIENT MEMBER

(76) Inventor: Alvaro Z. Gallegos, Albuquerque, NM (US)

Correspondence Address: LAW OFFICE OF RAY R. REGAN, P.A., P.O. BOX 1442, CORRALES, NM, 87048, US

(21) Appl. No.: 12/037,544

(22) Filed: Feb. 26, 2008

Related U.S. Application Data
(63) Continuation-in-part of application No. 12/018,358, filed on Jan. 23, 2008.

(51) Int. Cl. A43B 7/16; A43B 7/14; A61F 5/14

(52) U.S. Cl. 36/92; 36/88; 36/140

(57) ABSTRACT

The specification and drawing figures disclose, describe and claim a rigid orthotic plate with a resilient member, the combination positionable into footwear. In at least one aspect, a resilient member is fixedly mounted on the rigid orthotic. In another aspect, the resilient member is removably mountable on the rigid orthotic plate. In yet another aspect, a plurality of resilient members is provided.

### CROSS-REFERENCE TO RELATED APPLICATION

This application is a continuation-in-part from a co-pending application, Ser. No. 12/018,358 entitled REPLACEABLE HEEL SYSTEM, filed on Jan. 23, 2008 by the sole inventor named in this document. The specification of the pending application is incorporated by reference into this document.

### FIELD OF TECHNOLOGY

The apparatus and methods disclosed and claimed in this document pertain generally to footwear. More particularly, the new and useful rigid orthotic with resilient member disclosed and claimed in this document allows a footwear user to enjoy the pain relief and support provided by a rigid orthotic plate positionable in footwear, while also enjoying the comfort provided by a resilient member that is mountable in a chamber formed in the rigid orthotic.

### BACKGROUND

The evolution of the structure and design of shoes through the centuries has been and continues to be profound. Sandals were the most common footwear in early civilizations. Although footwear approximating a shoe began emerging in 1600 B.C., as late as 1850 A.D. most shoes were made on straight lasts (meaning that there was no difference between the right and left shoe). A “last” is the shaped block, usually made from wood, around which a shoe traditionally was designed and made. By 1892, the first rubber-soled shoes (called “plimsolls”) were manufactured in the United States. When vulcanization was discovered and patented by Charles Goodyear, rubber soled shoes became even more popular. These were followed by “sneakers” manufactured by U.S. Rubber using brand KEDS® in 1917. Beginning in 1958, an international demand arose for athletic shoes. Invention as a product of continuous research has progressed at an incredible pace.

For the longest time, shoes were merely functional; the primary objective was to protect feet. The idea of reducing or eliminating pain by redesigning footwear was a long time coming.

Studies show, however, that over 90 percent of people have different sized feet. A continual frustration has been the absence of a standard shoe size system. The problems, therefore, of fitting shoes to feet of a user often are multifactorial. Asymmetry and anatomical variation among feet provides significant challenges for people seeking to provide an ideal fit.

Until recently, however, few shoes addressed the problem of pain associated with walking, exercising, moving, or hiking, despite the fact that many people are unable to stand, move about, walk, or work in footwear generally available. Shoes were not designed to provide pain relief sufficient to enable users to walk and work. Many limitations that existed in the shoes provided by the footwear industry in connection with providing pain relief were overcome for countless people by the present inventor, Alvaro Z. Gallegos, by providing what is referred to generally as footwear that includes at least one compressible spring suspension system, such as the footwear disclosed and claimed in U.S. Pat. No. 5,435,079 issued on Jul. 25, 1995 to Gallegos, and in U.S. Design Pat. No. 434,548 issued Dec. 5, 2000 to Gallegos, and further protected under the internationally renown trademarks and service marks for the brand Z-COIL®.

Additional contributions to the art by the same inventor are disclosed and claimed in U.S. Pat. No. 5,970,630 issued on Oct. 26, 1999; U.S. Pat. No. 7,111,416 issued Sep. 26, 2006; U.S. Pat. No. 7,210,250 B2 issued on May 1, 2007; and application Ser. No. 11/697,735 filed on Apr. 8, 2007 for a Replaceable Heel System; and Application Ser. No. 12/018,358 filed on Jan. 23, 2008 for a Replaceable Heel System (collectively in this document, the “Prior Patents and Applications”).

Commercial embodiments of footwear based on the Prior Patents and Applications now contribute to relieving pain by providing in one or more embodiments, among other features disclosed and claimed in the Prior Patents and Applications, at least one spring, such as a coil spring, that provides superior support and high energy return. The spring, or coil spring (collectively, “spring”) disposed adjacent to a human heel and the heel of footwear, prevents bottoming out during compression during movement of a user. Commercial embodiments of footwear based on the Prior Patents and Applications also provide a stable and comparatively inexpensive footwear that incorporate and provides shock absorption and energy return during use of the footwear. Other commercial embodiments of footwear based on the Prior Patents and Applications also provide one or more midsoles, or plates, in the form of a rigid orthotic plate. The rigid orthotic plate extends from at least the metatarsal area of a user's foot to the rearmost portion of a user's foot (human heel), and also extends across the width of a user's foot, thus supporting the entire foot between the metatarsal area and the rear or heel portion of a foot. Cushioning in the form of a pad extending substantially across the upper surface of the rigid orthotic plate results, in combination, in a strong, stable and comfortable support for a user's foot.

Problems solved by the replaceable heel system of the parent application include at least providing a user the ability to extend the life of a shoe by replacing one replaceable heel with another replaceable heel. In at least one embodiment of the parent application, the replaceable heel system is provided with a rigid orthotic plate to cup or hold a human heel in place during use, thus distinguishing the replaceable heel system from other footwear in the industry. Currently available orthotic members are malleable, soft, and consequently offer little or no support.

A typical foot includes twenty-six bones. Bones are fragile. There is little distance between bones. Accordingly, a foot needs considerable support from footwear not only for use and functionality, but to relieve pain during use. The best support is rigid rather than flexible. Bones of a foot exposed to considerable impact and pressure during use are supported by a combination of a rigid orthotic plate and cushioning provided by a cushioning pad.

While the apparatus and methods disclosed and claimed in the parent application have proven useful for the intended applications described in that document, additional contributions to the art disclosed, shown and claimed in this document provide additional optimizations and embodiments in which the principles of operation and differing configurations result in additional features and uses for the rigid orthotic with resilient member disclosed, illustrated and claimed in this document.

The rigid orthotic with a resilient member disclosed and claimed in this document makes further contributions to the art by providing a variety of useful embodiments for enhancing pain relief, supporting a user's foot, and providing comfort through use of the resilient member. The result is an orthotic plate that distributes weight of the user both longitudinally and laterally, and provides cushioning for a foot.

### SUMMARY

The rigid orthotic with a resilient member, in one aspect, includes a resilient member that is permanently affixed within a substantially hollow chamber that is formed in the rigid orthotic. In another aspect, the resilient member may be removably inserted into the substantially hollow chamber formed in the rigid orthotic. In yet another aspect, a plurality of resilient members is provided, variably dimensioned, that may be inserted into variably dimensioned substantially hollow chambers either alone or in combination with one or more resilient members.

It will become apparent to one skilled in the art that the claimed subject matter as a whole, including the structure of the apparatus, and the cooperation of the elements of the apparatus, combine to result in a number of unexpected advantages and utilities. The advantages of the structure and co-operation of structure of the rigid orthotic with a resilient member will become apparent to those skilled in the art when read in conjunction with the following description, drawing figures, and appended claims.

The foregoing has outlined broadly the more important features of the invention to better understand the detailed description that follows, and to better understand the contributions to the art. The rigid orthotic with a resilient member is not limited in application to the details of construction, or to the arrangements of the components, provided in the following description or drawing figures, but is capable of other embodiments, and of being practiced and carried out in various ways.

The phraseology and terminology employed in this disclosure are for purposes of description, and therefore should not be regarded as limiting. As those skilled in the art will appreciate, the conception on which this disclosure is based readily may be used as a basis for designing other structures, methods, and systems. The claims, therefore, include equivalent constructions.

Further, the abstract associated with this disclosure is intended neither to define the replaceable heel system, which is measured by the claims, nor intended to limit the scope of the claims.

The novel features of the rigid orthotic plate with a resilient member are best understood from the accompanying drawing, considered in connection with the accompanying description of the drawing, in which similar reference characters refer to similar parts, and in which:

### BRIEF DESCRIPTION OF THE DRAWING

FIG. 1A of the drawing is an exploded perspective view of a embodiment of a replaceable heel system illustrating use of a compressible spring suspension system as optional;

FIG. 1B is an exploded perspective view of another embodiment of a replaceable heel system also illustrating use of a compressible spring suspension system as optional;

FIG. 1C is a perspective view of one of the replaceable heels of the replaceable heel system shown in FIG. 1B in greater detail;

FIG. 1D is a perspective view of a human foot with an orthotic positioned below the human foot;

FIG. 1E is a perspective view of the assembled components of the replaceable heel system shown in FIG. 1A illustrating use of a compressible spring suspension system as optional;

FIG. 1F is side cross-sectional view of the assembled components of the replaceable heel system shown in FIGS. 1B and 1C illustrating use of a compressible spring suspension system as optional;

FIG. 2A is an exploded perspective view of another embodiment of a replaceable heel system illustrating use of a compressible spring suspension system as optional;

FIG. 2B is a perspective view, partially cut-away, of the replaceable heel system illustrated in FIG. 2A illustrating use of a compressible spring suspension system as optional;

FIG. 3A is a side perspective view of another embodiment of a replaceable heel system;

FIG. 3B is an exploded side perspective view of the replaceable heel system illustrated in FIG. 3A; and

FIG. 4A is a perspective view of a rigid orthotic with resilient member positionable in footwear;

FIG. 4B is an end view of a rigid orthotic with resilient member showing contours in the a rigid orthotic with resilient member;

FIG. 4C is a perspective view of a rigid orthotic with resilient member useable as footwear;

FIG. 4D is a side view of a rigid orthotic with resilient member useable as footwear;

FIG. 4E is a side view of a rigid orthotic with resilient member useable as footwear, a plurality of resilient members, and means for attaching the resilient members and mounting a resilient member in a substantially hollow chamber of the rigid orthotic; and

FIG. 4F is a perspective view of a rigid orthotic with resilient member in relationship to a human foot.

In the detailed description to follow, to the extent that the numerical designations in the drawing figures include lower case letters such as “a,b” such designations include multiple references, and the letter “n” in lower case such as “a-n” is intended to express a number of repetitions of the element designated by that numerical reference and subscripts.

### DETAILED DESCRIPTION

### Definitions

As used in this document, the term “footwear” means, in general, a durable covering made for covering the human foot, to include at least shoes, athletic shoes, boots, chopines, and platforms.

The term “compressible spring suspension system” means at least the apparatus that includes the coil spring as shown and claimed in U.S. Pat. No. 5,435,079 issued on Jul. 25, 1995 to Gallegos, and in U.S. Design Pat. No. 434,548 issued Dec. 5, 2000 to Gallegos, and may include one or more leaf springs.

The term “replaceable,” as in the term “replaceable heel,” means to substitute one heel for another, indicating that each such replaceable heel is demountably attachable to, and detachable from, footwear, allowing a user to change at least one replaceable heel for another replaceable heel.

Because the unmodified term “heel” may have at least two different meanings, as used in this document the term “replaceable heel,” means a structure removably attachable to footwear adjacent the back of a shoe or sole of footwear, whereas the term “human heel” as used in this document means the part of a human foot below the ankle and behind the arch.

The terms “integral” and “integrally formed” as used in this document means a method of manufacture and assembly that includes “monolithic” and “unitary,” and is intended to be broad enough to not be limited to “one piece,” but sufficiently broad enough to embrace construction of the unit by means of, for example, uniting laminations or pieces of material by any means for fastening, including, but not limited to, interconnecting the component pieces by welding and/or connectors such as rivets or screws. The term “integral,” therefore, is intended to be broad enough to include any means of manufacture that maintains parts in a fixed relationship as a single unit, whether or not originally formed as a single unit, so as to work together as a single complete piece or unit, and be incapable of being easily dismantled without affecting the integrity of the piece or unit.

The term “upper” means the top most part of footwear such as a shoe and, depending on type and style, may include components such as the toe cap, vamp, tongue, quarters, and back.

The term “resilient” means that the resilient member is at least compressible, capable of withstanding shock without permanent deformation or rupture, and tends to recover from compression or other forces applied to the member.

The term “rigid orthotic” and “rigid orthotic plate” are used interchangeably, and refer to apparatus insertable into footwear to support and/or brace joints, muscles and bones of a user of footwear during use.

### Description

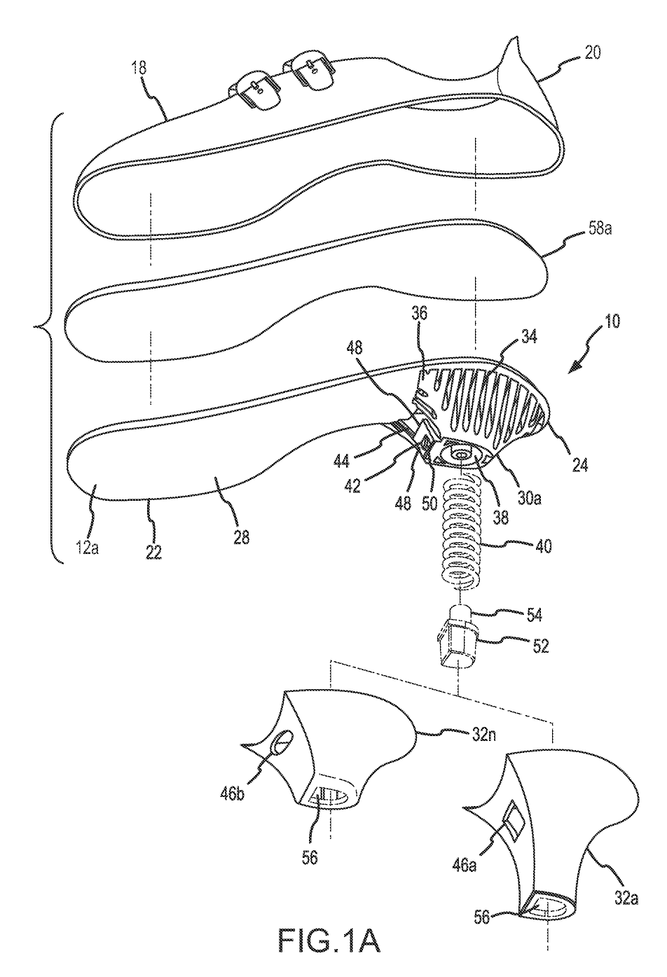

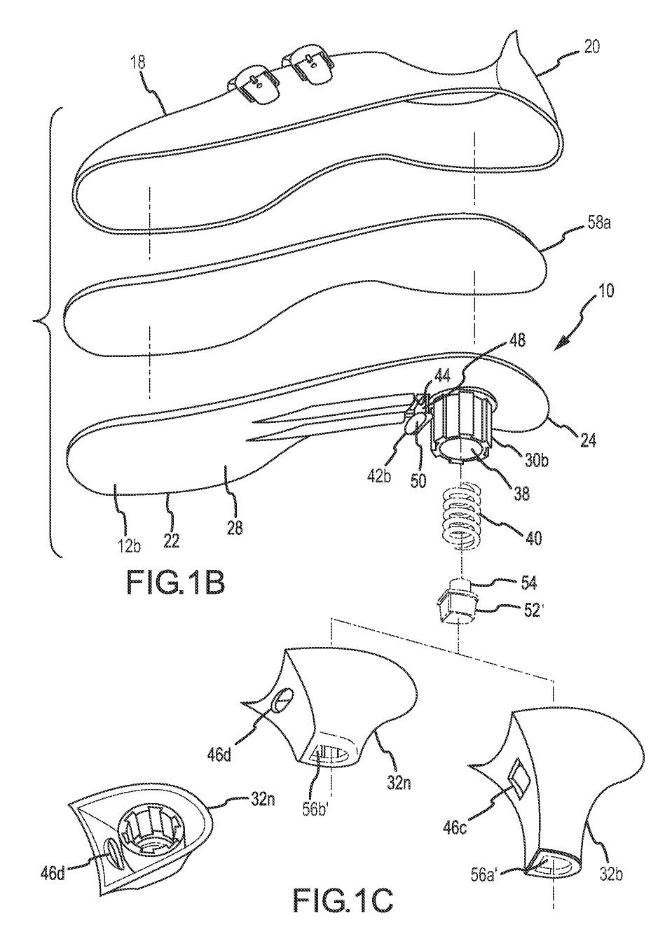

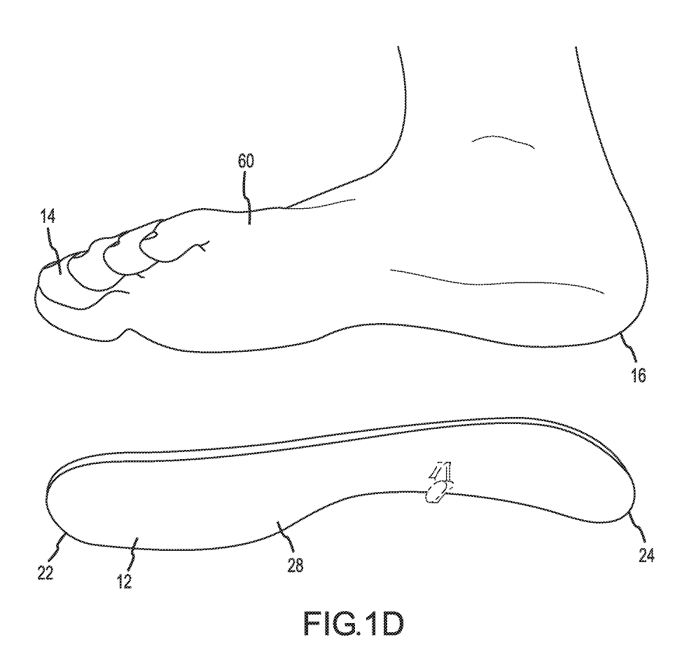

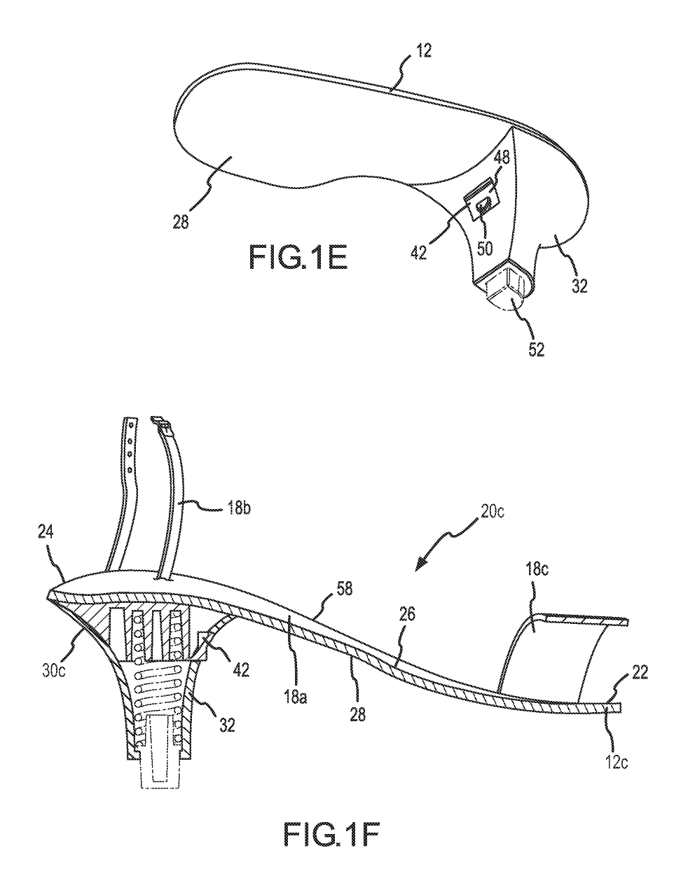

As illustrated in FIGS. 1A-3B, a replaceable heel system is provided that in its broadest context includes a plurality of replaceable heels and/or heel bodies. Means are provided to attach the plurality of replaceable heels to footwear that is adapted to demountably hold the plurality of heel bodies. In at least one embodiment, the plurality of heel bodies is connectable to an orthotic plate removably positionable in the footwear for distributing forces longitudinally and laterally during use of the footwear. In at least one other embodiment, a compressible spring suspension system is provided that is positionable in the plurality of replaceable heels to relieve impact pain during use of the footwear. However, use of a compressible spring suspension system in connection with the replaceable heel system is not a limitation of the replaceable heel system, as illustrated and claimed in this document.

More specifically, in the embodiment illustrated by cross-reference between FIGS. 1A-1F, a replaceable heel system 10 is illustrated that includes an integral contoured rigid orthotic plate 12. The integral contoured rigid orthotic plate 12 extends from a region adjacent to a plurality of human toes 14 to a region adjacent to a human heel 16 as perhaps best shown in by cross-reference between FIGS. 1A, 1B, and 1D. The integral contoured rigid orthotic plate 12 preferably is made from material selected from the group of materials consisting of one or more resins, plastic, metal, rigid rubber, metal alloys, and/or vinyl.

As also shown by cross reference between FIGS. 1A-1F, the integral contoured rigid orthotic plate 12 is positionable in at least an upper 18 of footwear 20, as shown in FIG. 1F. The integral contoured rigid orthotic plate 12 is formed with a leading end 22, a trailing end 24, a top surface 26, and a lower surface 28, as illustrated by cross-reference between FIGS. 1A-1F. As indicated, the orthotic plate 12 is rigid. Rigidity of the integral contoured rigid orthotic plate contributes to independently supporting each foot of a user during use, which has been proven to help reduce or eliminate pain experienced by those who wear footwear constructed of conventional materials and manufactured in conventional ways. Except as improved by the Prior Patents and Applications identified in this document, conventional footwear is composed of soft, resilient materials that create the illusion of comfort, but fail to address pain relief afforded by the footwear disclosed, illustrated and claimed in the Prior Patents and Applications, and in this document.

As also shown by cross-reference between FIGS. 1A-1F, a substantially inelastic housing 30 is included. The substantially inelastic housing 30a,b is integrally formed and mounted on the lower surface 28 of the integral contoured rigid orthotic plate 12. The substantially inelastic housing 30 is positioned adjacent the trailing end 24 of the integral contoured rigid orthotic plate 12. The substantially inelastic housing 30a,b is formed and designed to be removably engageable with a replaceable heel 32a-n.

In the embodiment shown in FIG. 1A, the substantially inelastic housing 30 includes a plurality of grooves 34. As shown, plurality of the grooves 34 is provided to achieve at least the following mechanical advantages: the plurality of grooves 34 in the exterior surface 36 of the housing 30a is adapted to reduce the weight of the footwear 20, to provide for slidable engagement of the substantially inelastic housing 30a with the plurality of substantially inelastic replaceable heels 32a-n, while maintaining rigidity of support between the substantially inelastic housing 30a and the substantially inelastic replaceable heels 32a-n. The substantially inelastic housing 30a also includes a bore 38. In an embodiment in which a compressible spring 40 is desired, bore 38 is provided to receive at least one end of a compressible spring 40. As indicated by broken lines for illustrating compressible spring 40, however, use of a compressible spring in connection with the replaceable heel system 10 is not a limitation of the replaceable heel system 10, as illustrated and claimed in this document.

In addition, in the embodiments shown by cross-reference between FIGS. 1A, 1C, 1E, and 1F, the replaceable heel system 10 includes a lock-and-release device 42. The lock-and-release device 42 is mounted on the integral contoured rigid orthotic plate 12. The lock-and-release device 42 is included to provide for removable attachment of the plurality of the replaceable heels 32a-n to the substantially inelastic housing 30. More specifically, the lock-and-release device 42 includes at least one aperture 44 formed in the integral contoured rigid orthotic plate 12.

In one embodiment, the lock-and-release device 42 includes an opening 46 formed in the plurality of substantially inelastic replaceable heels 32a-n. The shape and configuration of opening 46 is not a material feature of replaceable heel system 10. The lock-and-release device 42 further comprises a lever 48. The lever 48 includes a lip 50. The lever 48 flexibly extends from the aperture 44 formed in the integral contoured rigid orthotic plate 12. The lever 48, in combination with the lip 50, is adapted to retractably engage the opening 46 formed in the substantially inelastic replaceable heels 32a-n for removably attaching the plurality of replaceable heels 32a-n on footwear 20.

In another embodiment, shown perhaps best by cross-reference between FIGS. 1A-1B, the lever 48 and lip 50 of the lock-and-release device 42 are mountable on the substantially inelastic housing 30 to engage the opening 46 formed in the plurality of replaceable heels 32a-n to repositionably attach a replaceable heel 32a-n on footwear 20.

A compressible plunger 52 also is provided as illustrated in FIGS. 1A-1B. The compressible plunger 52 is positionable in the plurality of replaceable heels 32a-n, and in combination with compressible spring 40, provides resilient support during use of the footwear 20. The compressible plunger 52 is formed with a neck 54 adapted to engage one end of the compressible spring 40. In addition, an orifice 56 is provided. Orifice 56 is provided in replacable heels 32a-n. The orifice 56 is adapted to slidably extend and retract a portion of the compressible plunger 52 as shown in FIGS. 1A, 1B, and 1F. As indicated by broken lines for illustrating compressible spring 40, as well for orifice 56, however, use of a compressible spring in connection with the replaceable heel system 10 is not a limitation of the replaceable heel system 10, as illustrated and claimed in this document.

In addition, as shown in FIG. 1F, a resilient pad 58 is included. The resilient pad 58 is replaceably mountable on the top surface 26 of the integral contoured rigid orthotic plate 12. The resilient pad 58 provides cushion to a user's foot 60 during use of the footwear 20. The resilient pad 58 may be made of a variety of materials, especially foamed materials which have elastic or rebounding properties, such as materials comprised of silicon, neoprene, natural rubber foams, synthetic rubber foams and polyurethane, polyether and polyester foams, neoprene, Vinyl Nitrile, Styrene-Butadiene Rubber (SBR), Polyethylene (PE), ethel vinyl acetate (EVA), ethylene propylene terpolymer (EPT), EPT/PE/Butyl Rubber, Neoprene/EPT/SBR, epichlorohydrin (ECH), and nitrile (NBR) or a combination thereof or other cushioning materials known or used by one skilled in the art. The density and cell characteristics of the padding of the foam material are believed to be not material features in terms of providing the appropriate cushioning and rebound characteristics for cushioning, and may vary depending upon the type of activity of footwear in question.

The resilient pad 58 may have low to medium density to enhance deformability of the resilient pad 58. A low-density padding comprises material within the range of about 0.08 g/cm³ to about 0.50 g/cm³ An even more preferred range of densities for padding is material between about 0.1 g/cm³ to 0.30 g/cm³.

The resilient pad 58 also may be constructed of a closed-cell foam material, having a density in the range of about 0.08 g/cm³ to 0.50 g/cm³ , or of other suitable densities known to one skilled in the art. Alternatively, open-cell foam material, having a density in the range of about 0.08 g/cm³ to 0.40 g/cm³ may be used, or of other suitable densities known to one skilled in the art.

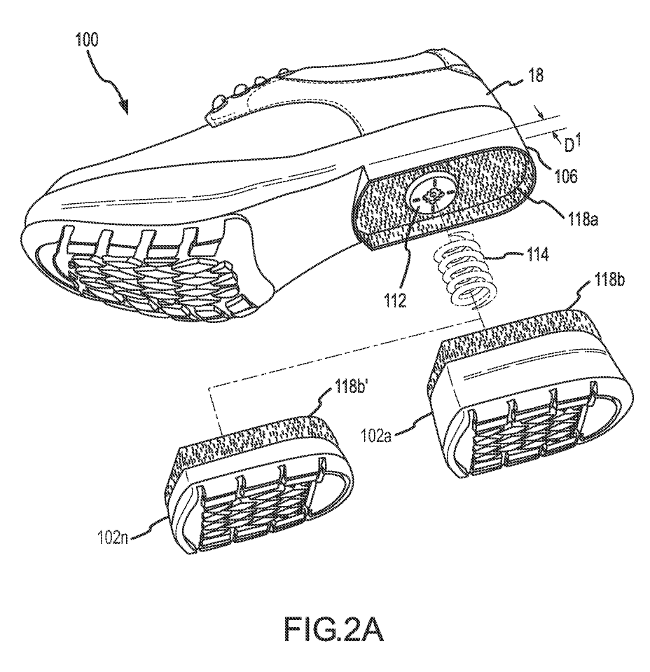

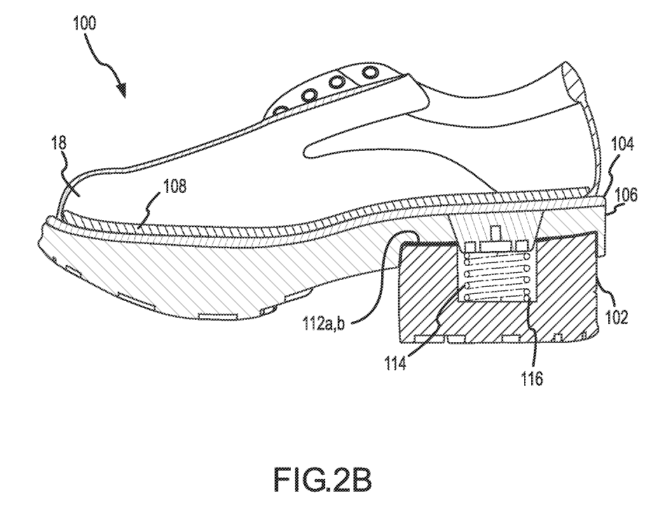

In the embodiment illustrated in FIGS. 2A-2B illustrates one of a plurality of replaceable heels 102. The footwear 100 with a plurality of replaceable heels 102 includes a contoured rigid orthotic plate 104 integrally formed with a housing 106 adapted for member ion into the footwear 100. In addition, a resilient pad 108 is replaceably mountable on the contoured rigid orthotic plate 104 to cushion a foot 60 during use of the footwear 100.

A substantially inelastic housing 110 is included. Substantially inelastic housing 110 is defined by the distance D¹ in FIG. 2A. The substantially inelastic housing 110 includes a plate 112 adapted to receive one end of a compressible spring 114. The housing 106 is attachable to the contoured rigid orthotic plate 104 to removably hold one of a plurality of replaceable heels 102. In addition, at least one compressible spring 114 is provided. The at least one compressible spring 114 is removable positionable in the housing 106 and in the plurality of replaceable heels 102. As indicated by broken lines for illustrating compressible spring 114, however, use of a compressible spring 114 in connection with the replaceable heel system 10 is not a limitation of the replaceable heel system 10, as illustrated and claimed in this document. A cavity 116 formed to receive the other end of the at least one compressible spring 114 is formed in the plurality of replaceable heels 102.

In the embodiment shown by cross-reference between FIGS. 2A-2B, opposing pressure-connectable and detachable members 118a,b are provided. The opposing pressure connectable and detachable members 118a,b are mounted on a plurality of replaceable heels 102 and on housing 106 to demountably connect the plurality of replaceable heels 102 to footwear 100. The opposing pressure-connectable and detachable members 118A,B are made from material selected from the group of materials consisting of hook-and-loop type fasteners such as Velcro®.

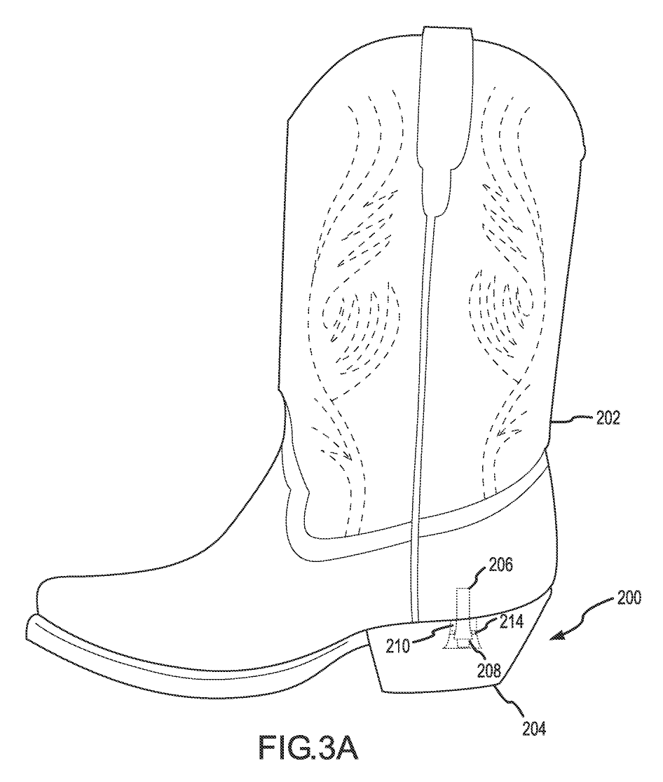

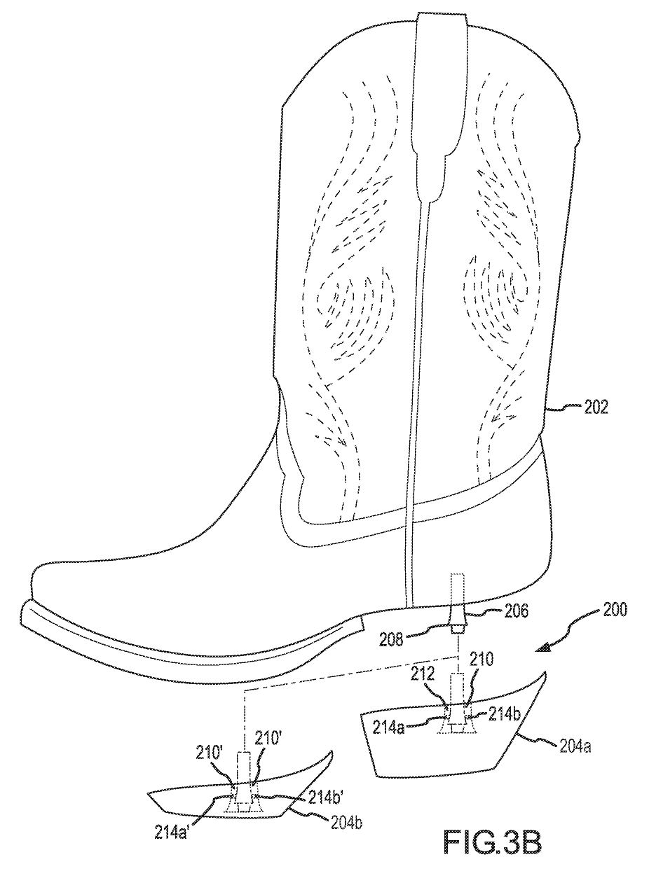

Another embodiment of a replaceable heel system is illustrated in FIGS. 3A-3B. As shown, a system for interchangeably replacing heels 200 on footwear 202 is shown. As shown, the system for interchangeably replacing heels 200 on footwear 202 includes a plurality of heel bodies 204. The one of a plurality of heel bodies 204 shown may be made of any material.

At least one flared plug 206 is provided. The at least one flared plug 206 is formed with an edge 208. As shown, the flared plug 206 is mountable in a duct 210 formed in the plurality of heel bodies 204 of the footwear 202. Material used to manufacture the at least one flared plug 206 is not a material consideration in the disclosure of this document, but preferably is formed from a plastic or resin material.

In addition, a substantially hollow receptacle 210 is included. The substantially hollow receptacle 210 is formed in the plurality of heel bodies 204 of the footwear 202. The substantially hollow receptacle 210 is formed with an inner surface 212. The inner surface 212 is dimensioned for slidable engagement with the flared plug 206 and compressible engagement with edge 208.

A plurality of peripherally mounted protrusions 214 is included. The plurality of peripherally mounted protrusions 214 is formed on the inner surface 212 of the hollow receptacle 210, and extends toward the longitudinal axis of the substantially hollow receptacle 210. Each of the peripherally mounted protrusions 214 is provided to perform the mechanical advantage of removably connecting the flared plug 206 and the substantially hollow receptacle 210. More specifically, as illustrated in FIGS. 3A-3B, in operation the at least one flared plug 206 is removably connectable to the substantially hollow receptacle 210 by application of pressure by the user of the footwear by applying hand pressure on the replaceable heel bodies 204 so as to direct the substantially hollow receptacle 210 against the flared plug 206.

While the apparatus, system, and methods disclosed above have proven useful for the intended applications described in that document, additional contributions to the art disclosed, shown and claimed in this document provide additional optimizations and embodiments in which the principles of operation and differing configurations result in additional features and uses resulting in the rigid orthotic with resilient member disclosed, illustrated, and claimed in this document.

More specifically, the new and useful rigid orthotic with resilient member disclosed and claimed in this document allows a footwear user to enjoy the pain relief and support provided by a rigid orthotic plate positionable in footwear, while also enjoying the comfort of a resilient pad.

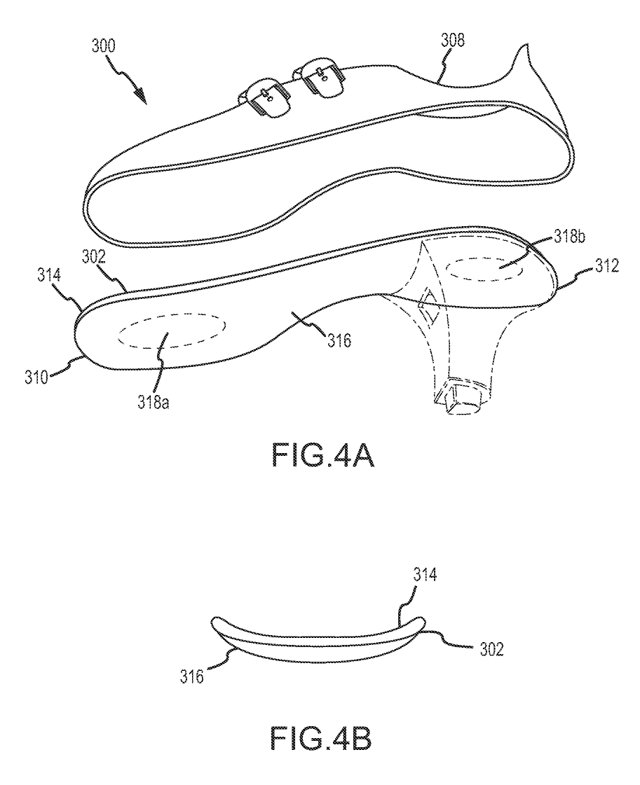

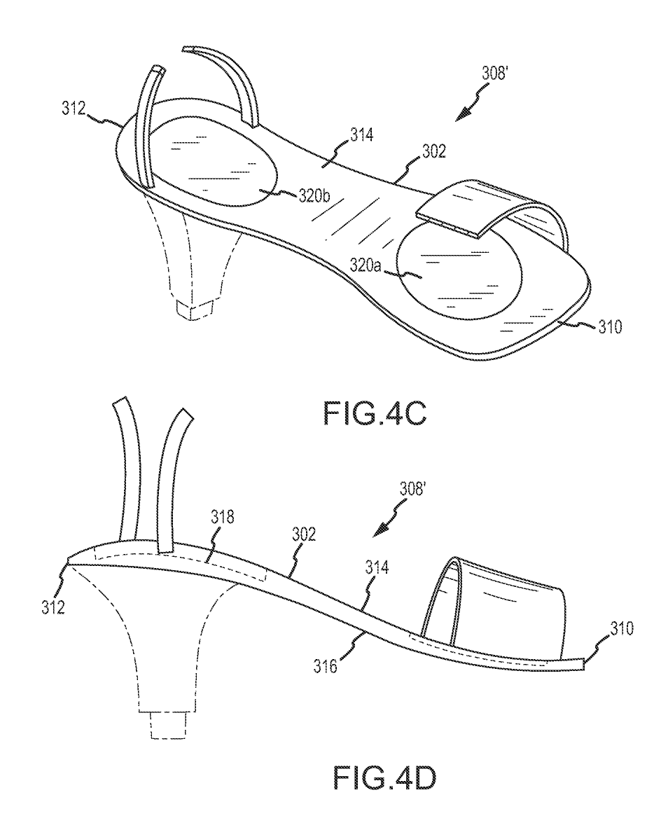

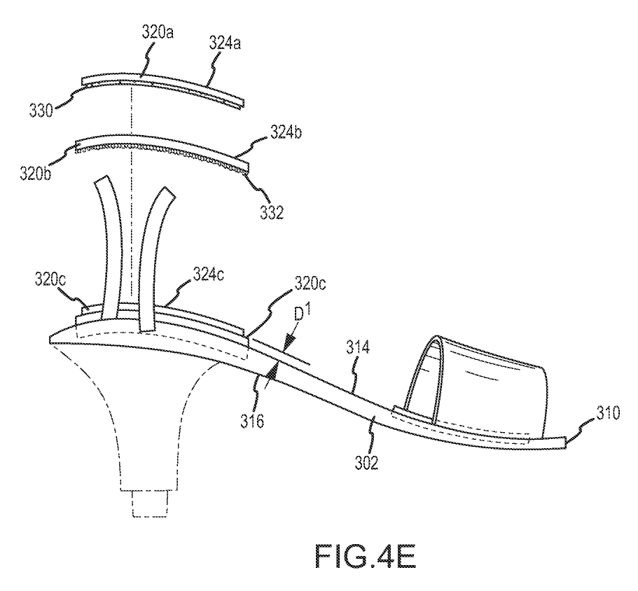

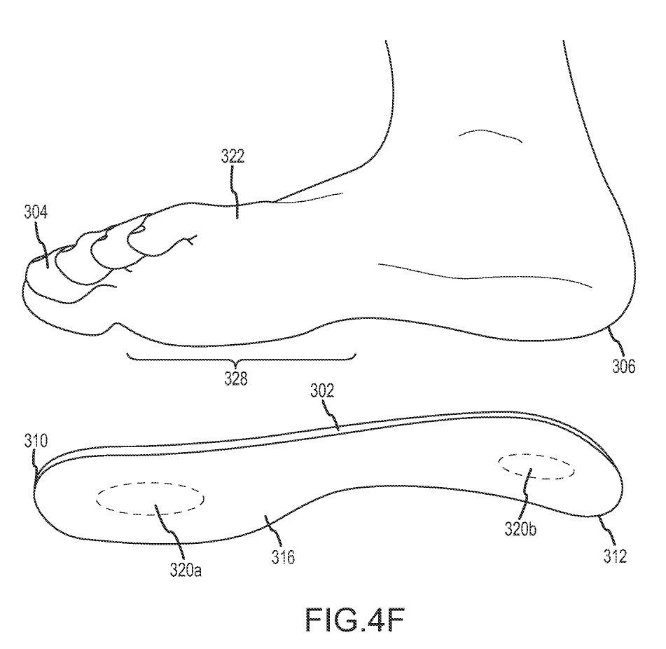

More particularly, as illustrated by cross-reference between FIGS. 4A-4F, a rigid orthotic with resilient member is shown. As illustrated, the rigid orthotic with resilient member 300 includes a rigid orthotic plate 302 extending from a region adjacent to a plurality of human toes 304 to a region adjacent to a human heel 306 adapted for positioning in footwear 308. As also illustrated, the rigid orthotic with resilient member 300 is preferably an integral contoured rigid orthotic plate 302, the area of the contours being identified by the legend “contour” in FIG. 4B. As also illustrated by cross-reference between FIGS. 4A-4F, the rigid orthotic with resilient member 300 is formed with a leading end 310, a trailing end 312, a top surface 314, and a lower surface 316.

In addition, as perhaps best shown by cross-reference between FIGS. 4A and 4C-4D, a substantially hollow chamber 318 is formed in the rigid orthotic plate 302 adapted to receive insertion of a resilient member 320 for cushioning portions of a foot 322 during use of the footwear 308. As illustrated in FIGS. 4C-4D, however, the rigid orthotic plate itself may constitute the footwear 308′. The resilient member 320 is shaped and dimensioned to have a distal side 324 and a proximal side 326. As illustrated in FIG. 4C, in one aspect of the rigid orthotic with resilient member 300, the proximal side 326 of the resilient member 320 does not extend above the plane of the top surface 314 of the rigid orthotic plate 302 following insertion of resilient member 320 into the substantially hollow chamber 318. As illustrated in FIG. 4E, in another aspect of the rigid orthotic with resilient member 300, the proximal side 324 of the resilient member 320 does extend a predetermined distance D¹ above the plane of the top surface 314 of the rigid orthotic plate 302 following insertion of resilient member 320 into the substantially hollow chamber 318. As also illustrated in FIG. 4E, in yet another aspect of the rigid orthotic with resilient member 300, a plurality of resilient members 320a-c for cushioning portions of a foot 322 during use of the footwear 308 and 308′ are dimensioned and sized for being insertable into resilient member 320, in which circumstance the proximal side 324a, for example, of resilient member 320a may or may not extend a predetermined distance D¹ above the top surface 314 of the rigid orthotic plate 302 following insertion of resilient member 320 into the substantially hollow chamber 318.

The rigid orthotic plate 302 is made from one or more materials selected from the group consisting of resins, plastic, metal, rigid rubber, metal alloys, and/or vinyl.

Resilient member 320 is made from one or more foamed materials. In addition, materials selected for making the resilient member 320 may include materials having elastic or rebounding properties. Accordingly, materials selected for making the resilient member 320 may include materials such as silicon, neoprene, natural rubber foams, synthetic rubber foams and polyurethane, polyether and polyester foams, neoprene, Vinyl Nitrile, Styrene-Butadiene Rubber (SBR), Polyethylene (PE), ethyl vinyl acetate (EVA), ethylene propylene terpolymer (EPT), EPT/PE/Butyl Rubber, Neoprene/EPT/SBR, epichlorohydrin (ECH), and nitrile (NBR).

The materials chosen for making the resilient member 320 may include low to medium density materials to enhance deformability of resilient member 320. Thus, the resilient member 320 may be made of low-density padding materials within the range of about 0.08 g/cm³ to about 0.50 g/cm³, and/or materials having a low-density padding within the range of between about 0.1 g/cm³ to 0.30 g/cm³ . Likewise, as a person skilled in the art will appreciate, the resilient member 320 may be made of closed-cell or open-cell foam materials.

To achieve the goals of providing comfort and cushioning to a user of footwear 308, the resilient member 320, as illustrated by cross-reference between FIGS. 4E and 4F, is positionable substantially beneath a metatarsal region 328 of a human foot 322. Either alone or in combination with resilient member 320a positioned in substantially hollow chamber 318 substantially beneath a metatarsal region 328 of a human foot 322, resilient member 320b may be positioned in substantially hollow chamber 318 substantially beneath a human heel 306.

As also illustrated in FIG. 4E, the resilient member 320 may be fixedly inserted into the substantially hollow chamber 318. As a person skilled in the art will appreciate, a resilient member 320 may be fixedly inserted into the substantially hollow chamber 318 using an adhesive 330, illustrated by cross-hatching for descriptive purposes, and/or a hook-and-loop material 332, as shown in FIG. 4E. The hook-and-loop material 332, as shown in FIG. 4E, may also be used to make resilient member 320 removably insertable into the substantially hollow chamber 318. Likewise, a plurality of resilient members 320a-c, as illustrated in FIG. 4E, may be combined using hook-and-loop material 332, and the combination inserted into substantially hollow chamber 318.

As a person skilled in the art will appreciate, the size, shape, and dimensions of a resilient members 320 and of a substantially hollow chamber 318 are not material considerations of the rigid orthotic with resilient member 300 disclosed, illustrated, and claimed in this document.

Claim elements and steps in this document have been numbered solely as an aid in understanding the description. The numbering is not intended to, and should not be considered as intending to, indicate the ordering of elements and steps in the claims. In addition, the rigid orthotic with resilient member illustrated in drawing FIGS. 4A through 4F show at least one embodiment that is not intended to be exclusive, but merely illustrative of the disclosed embodiments.

What is claimed is:

1. A rigid orthotic with resilient member, comprising:
an integral contoured rigid orthotic plate extending from a region adjacent to a plurality of human toes to a region adjacent to a human heel adapted for positioning in footwear,
wherein the integral contoured rigid orthotic plate is formed with a leading end, a
trailing end, a top surface, and a lower surface;
a substantially hollow chamber formed in the integral contoured rigid orthotic plate adapted to receive insertion of a resilient member; and
a resilient member insertable into the substantially hollow chamber adapted to cushion a foot during use of the footwear.

2. A rigid orthotic with resilient member as recited in claim 1, wherein the integral contoured rigid orthotic plate is made from one or more materials selected from the group consisting of resins, plastic, metal, rigid rubber, metal alloys, and/or vinyl.

3. A rigid orthotic with resilient member as recited in claim 1, wherein the resilient member is made from one or more foamed materials.

4. A rigid orthotic with resilient member as recited in claim 1, wherein the resilient member is made from one or more materials selected from the group consisting of materials having elastic or rebounding properties.

5. A rigid orthotic with resilient member as recited in claim 1, wherein the resilient member is made from one or more materials selected from the group consisting of silicon, neoprene, natural rubber foams, synthetic rubber foams and polyurethane, polyether and polyester foams, neoprene, Vinyl Nitrile, Styrene-Butadiene Rubber (SBR), Polyethylene (PE), ethyl vinyl acetate (EVA), ethylene propylene terpolymer (EPT), EPT/PE/Butyl Rubber, Neoprene/EPT/SBR, epichlorohydrin (ECH), and nitrile (NBR).

6. A rigid orthotic with resilient member as recited in claim 1, wherein the resilient member is made from one or more materials selected from the group of materials consisting of low to medium density materials adapted to enhance deformability.

7. A rigid orthotic with resilient member as recited in claim 1, wherein the resilient member is made of materials having a low-density padding within the range of about 0.08 g/cm³ to about 0.50 g/cm³.

8. A rigid orthotic with resilient member as recited in claim 1, wherein the resilient member is made of materials having a low-density padding within the range of between about 0.1 g/cm³ to 0.30 g/cm³.

9. A rigid orthotic with resilient member as recited in claim 1, wherein the resilient member is made of one or more closed-cell foam materials.

10. A rigid orthotic with resilient member as recited in claim 1, wherein the resilient member is made of one or more open-cell foam materials.

11. A rigid orthotic with resilient member as recited in claim 1, wherein the resilient member is shaped and dimensioned to have a distal side and a proximal side, and further wherein the proximal side of the resilient member after insertion into the substantially hollow chamber does not extend above the plane of the top surface of the integral contoured rigid orthotic plate.

12. A rigid orthotic with resilient member as recited in claim 11, wherein the proximal side of the resilient member after insertion into the substantially hollow chamber protrudes a predetermined distance above the top surface of the integral contoured rigid orthotic plate.

13. A rigid orthotic with resilient member as recited in claim 1, wherein the resilient member is positionable substantially adjacent to and beneath a metatarsal region of a human foot.

14. A rigid orthotic with resilient member as recited in claim 1, wherein the resilient member is positionable substantially adjacent to and beneath a human heel.

15. A rigid orthotic with resilient member as recited in claim 1, wherein the resilient member is fixedly inserted into the substantially hollow chamber.

16. A rigid orthotic with resilient member as recited in claim 1, wherein the resilient member is removably insertable in the substantially hollow chamber.

17. An orthotic with resilient member, comprising:
a rigid orthotic plate for use as footwear;
a plurality of substantially hollow chambers formed in the rigid orthotic plate for holding one or more resilient members; and
one or more resilient members insertable into the plurality of substantially hollow chambers.

18. An orthotic with resilient member as recited in claim 17, wherein the one or more resilient members is shaped and dimensioned for insertion into the plurality of substantially hollow chambers.

19. An orthotic with resilient member as recited in claim 18, wherein the one or more resilient members is shaped and dimensioned for insertion into the plurality of substantially hollow chambers.

20. An orthotic with resilient member as recited in claim 17, further comprising means for affixing the one or more resilient members in the plurality of substantially hollow chambers.

21. An orthotic with resilient member as recited in claim 17, further comprising means for removably inserting the one or more resilient members in the plurality of substantially hollow chambers.

22. A method of making an orthotic for use with footwear, comprising:
selecting a first material for shaping a rigid orthotic plate positionable in the footwear;
manipulating the material to form the rigid orthotic plate;
forming in the rigid orthotic plate one or more substantially hollow chambers dimensioned to receive one or more resilient members;
choosing a second material to form a resilient member;
forming the one or more resilient members;
inserting the one or more resilient members into the one or more substantially hollow chambers; and
disposing the orthotic in the footwear.

23. A method of making an orthotic for use with footwear as recited in claim 22, wherein the step of selecting a first material for shaping a rigid orthotic plate positionable in the footwear includes the substep of selecting one or more materials selected from the group consisting of resins, plastic, metal, rigid rubber, metal alloys, and/or vinyl.

24. A method of making an orthotic for use with footwear as recited in claim 22, wherein the step of choosing a second material to form a resilient member includes the substeps of:
a) choosing a material from the group of materials consisting of low to medium density materials to enhance deformability;
b) choosing a material having a low-density padding within the range of about 0.08 g/cm³ to about 0.50 g/cm³;
c) choosing a material having a low-density padding within the range of between about 0.1 g/cm³ to 0.30 g/cm³;
d) choosing one or more closed-cell foam materials; and/or e) choosing one or more open-cell foam materials.
